(19) United States
(12) Patent Application Publication
(10) Pub. No.: US 2024/0274035 A1
(43) Pub. Date: Aug. 15, 2024

(54) SYSTEM AND METHOD FOR DETERMINING THE DISTRIBUTION OF FORCES ALONG THE BLADE OF A LARYNGOSCOPE

(71) Applicant: CAE HEALTHCARE CANADA INC., Saint-Laurent (CA)

(72) Inventors: Mathieu LEONARD, St-Remi (CA); Laurent DESMET, Toulouse (FR)

(21) Appl. No.: 18/181,793

(22) Filed: Mar. 10, 2023

(30) Foreign Application Priority Data
Feb. 14, 2023 (CA) 3189567

(51) Int. Cl. G09B 23/28; G16H 40/63

(52) U.S. Cl. CPC G09B 23/285; G16H 40/63; A61B 1/267

(57) ABSTRACT

A training platform, a method and a non-transitory storage medium are provided to evaluate the performance of a user in performing a laryngoscopy, using a force-sensor equipped laryngoscope.

### TECHNICAL FIELD OF THE INVENTION

The present invention relates to the field of healthcare training systems and methods, and more specifically relates to systems and methods for providing feedback to users in manipulating a laryngoscope during laryngoscopy.

### BACKGROUND

Training or evaluating users in performing laryngoscopies is extremely challenging since there is limited information on which trainers or clinicians can rely to provide personalized and real-time feedback to improve intubation skills. Trainers cannot readily determine whether the pressure applied on the tongue, vallecula or epiglottis is adequate, to allow proper view or access to the larynx.

There have been some developments made to equip training laryngoscopes with force sensors in the handle. Such solutions capture an overall force applied on the blade, which is helpful, but insufficient to guide users in repositioning the laryngoscope or in modifying their grip.

There is therefore a need for a system and a method that would provide improved feedback to users when performing laryngoscopies.

### BRIEF SUMMARY OF THE INVENTION

According to an aspect, a laryngoscopy training system is provided. The laryngoscopy training system comprises a laryngoscope including a blade provided with a plurality of force sensors therealong. The force sensors are adapted to detect forces applied thereon resulting from a user inserting or manipulating the laryngoscope inside a mouth and an airway, and to transmit respective force signals. The laryngoscopy training system may also comprise a processing device comprising a processor and storage medium having stored thereon processor-readable instructions for processing force data derived from the respective force signals. The processor-readable instructions are also for determining a force distribution along the blade based on the force data; for comparing the force distribution determined with a reference force distribution; and for outputting for display a visual indication indicative of a deviation of the force distribution being applied along the blade from the reference force distribution, in real-time.

In possible embodiments, the force sensors are provided on a flexible sensor strip positioned on an inner surface of the blade devised to be in contact with airway structures.

In possible embodiments, the laryngoscopy training system further comprises a display for displaying a graphical user interface, the graphical user interface comprising the visual indication or being adapted to display the visual indication.

In possible embodiments, the laryngoscopy training system further comprises a printed circuit board (PCB) including a communication unit, the communication unit including input connections for receiving the respective force signals from the force sensors, and one or more output connection(s) for sending the force data via a wired or wireless connection to the processing device. The PCB may be provided on or within the laryngoscope.

In possible embodiments, laryngoscope reference force distribution data is stored onto the memory or storage medium of the processing device and the comparison is performed based on predetermined thresholds.

In possible embodiments, the visual indication comprises a representation of the blade. The force distribution and/or the deviation may be illustrated on or near the blade using color, icons, letters or numbers.

In possible embodiments, an on-board video camera can be provided on or near the blade, for capturing images during training sessions. The graphical user interface may further display the images captured, preferably in real-time, in addition to the visual indication of the force distribution and/or deviation from the reference force distribution.

In possible embodiments, the processing device may comprise a trained predictive model for recognizing a force distribution pattern applied by the user based on previously learned force distribution patterns derived from previously recorded force data. The processing device may be further configured for providing for output personalized feedback to the user regarding force adjustments needed to come closer to one of the previously learned patterns.

In possible embodiments, the processing device may be configured for storing several force distribution patterns associated with a user over time; and for providing, using the trained predictive model, an indication of the improvement of a performance of the user over time, in reaching a standard force distribution pattern.

In possible embodiments, the system can include an additional trained predictive model trained on previously captured laryngoscopy images labelled as valid or invalid, to determine whether the blade is properly positioned.

In possible embodiments, the additional trained predictive model can further determine a grade or degree of aperture of the larynx based on previously labelled laryngoscopy images.

In possible embodiments, the processing device is configured for processing in real time a plurality of time buffers, and for computing, for each time buffer, statistical data of the forces measured by each of the sensors during a predetermined period while an instructor or clinician performs a laryngoscopy using the laryngoscope. The trained predictive model can be configured to detect force distribution patterns along the blade using the statistical data computed for the plurality of time buffers.

In possible embodiments, the trained predictive model is a support-vector machine (SVM) model.

According to another aspect, a method is provided, for evaluating a performance of a user in performing a laryngoscopy. The method comprises measuring force signals associated to forces applied to different portions of a blade of a laryngoscope manipulated by a user during the laryngoscopy; converting the force signals into force data indicative of a distribution of forces along the blade; comparing the distribution of forces along the blade with at least one previously determined force distribution pattern; and providing for output, in real time, an indication of whether too little, adequate or too much force is applied to each of the portions of the blade, relative to the at least one previously determined force distribution pattern, while the user manipulates the laryngoscope.

In possible embodiments, the method may include a step of associating the distribution of forces applied by the user to a given force distribution pattern determined using a predictive model. This step may be part of comparing the distribution of forces along the blade with the previously determined force distribution pattern(s).

In possible embodiments, the method comprises a step of determining additional practice time required by the user to reach a standard force distribution pattern using the predictive model.

According to another aspect, a computer implemented method is provided, for configuring a laryngoscopy training system used for evaluating a performance of a user in performing a laryngoscopy. The method comprises measuring force signals with force sensors provided along a blade of a laryngoscope, while performing the laryngoscopy; processing time buffers associated with the force signals, each time buffer corresponding to a period of the laryngoscopy, each time buffer comprising statistical data derived from the force signals of each of the sensors; classifying the time buffers by assigning labels thereto, the labels providing an indication as to whether the force along the blade is properly distributed; and training a predictive model using the time buffers labelled to predict whether future captured time buffers correspond to a laryngoscopy being performed with a valid force distribution along the blade.

According to another aspect, a non-transitory storage medium for storing processor-executable instructions for evaluating a performance of a user in performing a laryngoscopy, the instructions causing a processor to: process force data derived from force signals indicative of forces applied to different portions of a blade of a laryngoscope manipulated by a user during the laryngoscopy; determine a distribution of forces along the blade; evaluate or compare the distribution of forces along the blade with/relative to at least one previously determined force distribution pattern; and provide for output, in real time, an indication of whether too little or too much force is applied to each of the portions of the blade, relative to the at least one previously determined force distribution pattern, while the user manipulates the laryngoscope.

In possible embodiments, the non-transitory storage medium further comprises instructions for causing the processor to: use a predictive model trained on previously collected force signals, collected during a valid laryngoscopy procedure. The non-transitory storage medium may also comprise instructions for causing the processor to determine additional practice time required by the user to reach a standard force distribution pattern using the predictive model.

### BRIEF DESCRIPTION OF THE DRAWINGS

Other objects, advantages and features will become more apparent upon reading the following non-restrictive description of embodiments thereof, given for the purpose of exemplification only, with reference to the accompanying drawings in which:

FIG. 1A is a schematic cross-sectional view of a patient undergoing a laryngoscopy with a curve-blade laryngoscope, where the tip of the blade is in contact with the vallecula. FIG. 1C is an enlarged view of a portion of FIG. 1A. FIG. 1B is another schematic cross-sectional view of a patient undergoing a laryngoscopy with a straight-blade laryngoscope, where the tip of the blade is in contact with the epiglottis. FIG. 1D is an enlarged view of a portion of FIG. 1B.

FIG. 2 shows a schematic view of laryngoscopy training platform, according to a possible embodiment. FIG. 2A is a schematic view of the laryngoscope of the laryngoscopy training system of FIG. 2, the blade of the laryngoscope being provided with force sensors therealong, according to a first embodiment.

FIG. 3 is a side view of a laryngoscope for a laryngoscopy training platform, according to a second embodiment.

FIG. 4 is a bottom view of a laryngoscope for a laryngoscopy training platform, showing the blade provided with force sensors, according to a third embodiment.

FIG. 5A is a side perspective view of a laryngoscope for a laryngoscopy training system, according to a fourth embodiment. FIG. 5B is another view of the laryngoscope of FIG. 5A, showing the onboard PCB.

FIG. 6 is a schematic view of a graphical user interface of the laryngoscopy training system, according to a possible embodiment.

FIG. 7 is a schematic diagram of a portion of the method for determining a force distribution pattern being applied by a user manipulating the laryngoscope, by a trained predictive model.

FIG. 8A is a flow chart showing steps for training a predictive model, part of the laryngoscopy training system, according to a possible implementation.

FIG. 8B is a flow chart showing steps for providing feedback to a user performing a laryngoscopy, as to whether too little or too much force is applied to each portion of the blade, relative to a previously determined force distribution pattern, according to a possible implementation.

### DETAILED DESCRIPTION

In the following description, the same numerical references refer to similar elements. The embodiments mentioned in the present description are embodiments only, given solely for exemplification purposes.

Moreover, although the embodiments of the method and system for evaluating the performance of a user in performing a laryngoscopy consist of certain configurations as explained and illustrated herein, not all these configurations are essential and should not be taken in their restrictive sense. It is to be understood, as also apparent to a person skilled in the art, that other suitable components and cooperations therebetween, as well as other suitable configurations, may be used for the method and system for performing evaluations, as will be briefly explained herein and as can be easily inferred herefrom by a person skilled in the art.

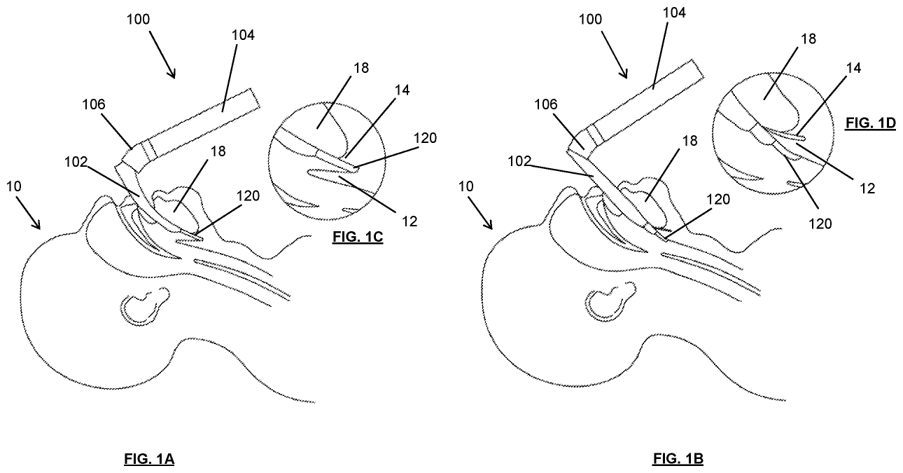

Referring to FIGS. 1A and 1B, two different types of laryngoscopes 100 are shown. A laryngoscope 100 generally comprises a blade 102 extending from a handle 104. Some models include a hinge connection 106 connecting the blade 102 to the handle 104. On FIGS. 1A and 1C, a curved-blade laryngoscope 100 is illustrated, where the underside of the blade 102 is configured to press against the tongue 18 and the tip 120 of the blade is curved and is configured to interact with the vallecula 14 of the patient 10, to provide access to the larynx. On FIGS. 1B and 1D, a straight-blade laryngoscope 100 is illustrated, where the flat underside of the blade 102 is configured to press against the tongue 18 and the tip 120 of the blade 102 is configured to interact with the epiglottis 12 of the patient, to provide access to the larynx. Curved-blades are sometimes referred to as “Macintosh” or “Mac” blades, while straight-blades are referred to as “Miller” blades. A curved-blade is typically positioned in the vallecula, to lift the epiglottis out of the airway, while a straight-blade is positioned on top of the epiglottis, to trap it and expose the glottis and the vocal cords. There also exists laryngoscopes that can be used for both techniques.

A laryngoscopy is a delicate operation, and can lead to complications when not performed properly, including pain, swelling, bleeding, gagging, teeth damage and infection. In addition, if a laryngoscopy is not performed correctly, avoidable surgeries may be performed while intubation would have been possible, had the laryngoscopy been conducted adequately.

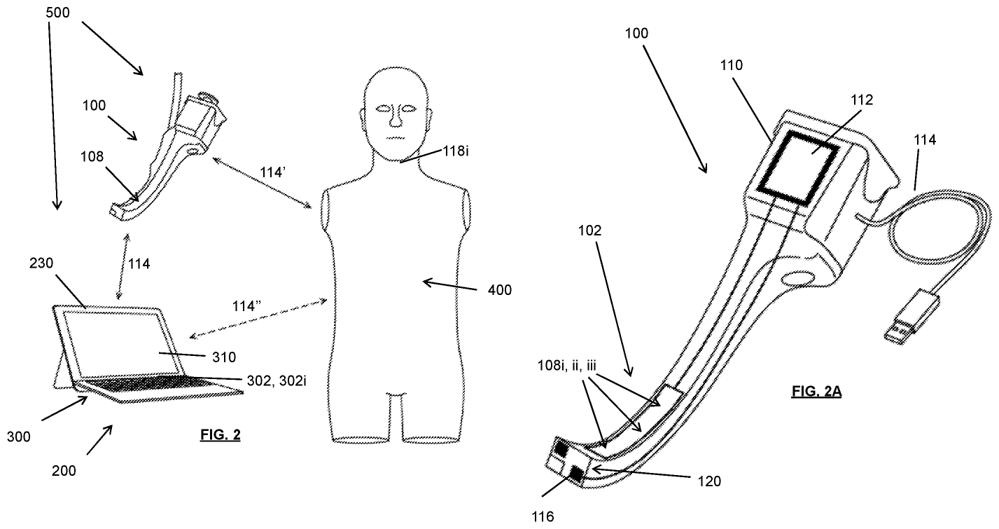

Referring to FIGS. 2 and 2A, a laryngoscopy training system 500 is schematically illustrated, according to a possible implementation. The training system 500 (which may also be referred to as a laryngoscopy evaluation system or a laryngoscopy training platform) comprises a force-sensor equipped laryngoscope 100, a processing device 200, and optionally, a training manikin 400. The processing device can consist in a single device or can be distributed among several devices. It may comprise one or more processors and includes storage medium. The storage medium includes non-volatile (ROM, flash, SSD, or other) and volatile (such as RAM) memory, which may be distributed or not. The training system 500 also includes a user interface 310 to provide feedback to users and/or trainers, as to whether a proper force distribution is being applied to the laryngoscope 100. The blade 102 of the laryngoscope 100 is provided with a plurality of force sensors 108 along the length of the blade. The force sensors 108 are adapted to detect respective forces applied thereon by a user when inserting or manipulating the laryngoscope inside a mouth and an airway of a patient or manikin, and to transmit respective force signals. While force sensors are used, it will be noted that pressure sensors could also be used. Force sensors are adapted to measure the force (typically in Newtons), while pressure sensors are adapted to measure a force over a surface (such as N/mm² or PSI), such as the surface of a sensor.

Referring to FIG. 2A, the portion of the blade 102 designed to contact the tongue of the patient (or equivalent structure in a training manikin) is thus divided in a plurality of blade regions or portions extending along the blade, each blade portion or region being provided with a corresponding force sensor 108i, 108ii, 108iii. Different forces can thus be detected along the blade, from the tip 120 toward the handle. It will be noted that the tip 120 has been enlarged to better illustrate the possibility of including an on-board camera 116, but the tip of the blade is generally thinner than the schematic illustration of FIG. 2A.

Referring to FIGS. 2 and 2A, the laryngoscope 100 is also provided with a communication unit 112 for receiving the force signals from the respective force sensors 108i-iii and for sending force data indicative of the force applied on each sensor 108. The communication unit can be any device or module that can transmit the force data for further processing, wirelessly or via wires, relying on technologies such as Wi-Fi, Bluetooth, Ethernet technologies, as examples only. A communication link 114 is also provided for sending the force data to the processing device. Additional communication links 114′, 114″ may also be established between the laryngoscope 100 and the training manikin 400 or between the training manikin and the processing device 200 (identified in FIG. 2). While the illustrated embodiment shows a wired link in FIG. 2A, wireless connections are also possible. Also of note, in the example illustrated, processing of the force data is mostly performed by the processing device 200, which in this case is a laptop, but it can be considered to have most or all of the force data processing performed by an on-board processor, on a printed circuit board 110, provided it has the required processing and storage capacity. In yet other embodiments, the processing device 200 may be distributed and may include remote servers, such as cloud-based servers, on which some or all of the processing can be performed.

The processing device 200 thus receives the force data and comprises a processor and a memory having stored thereon processor-readable instructions to determine a force distribution along the blade. The force distribution can be compared with a reference force distribution, previously determined, for example by evaluating or assessing the similarity of the measured force distribution with one or more reference force distribution(s). The user interface 310, which is preferably a graphical user interface, shows to the user a visual indication of how far or how close the force distribution being applied is to the reference force distribution. In other words, the user interface can indicate, using colors, text, numbers, etc., the deviation of the force distribution being applied from a reference force distribution, in real-time. The deviation can be expressed in different ways, such as a difference in newtons (N) or in pounds per square inch (psi) along the blade from the reference force distribution, as a percentage, with a grade or score, or using colors, as examples only. In the illustrated embodiment, the user interface is displayed by the display screen 230 of the laptop, but it can be considered to have the user interface displayed on a portable tablet, an intelligent phone or watch, or on a screen provided on the laryngoscope.

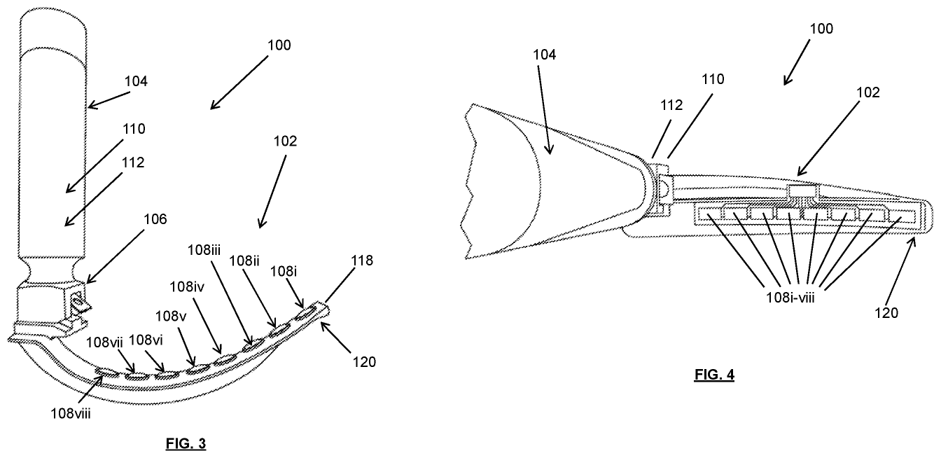

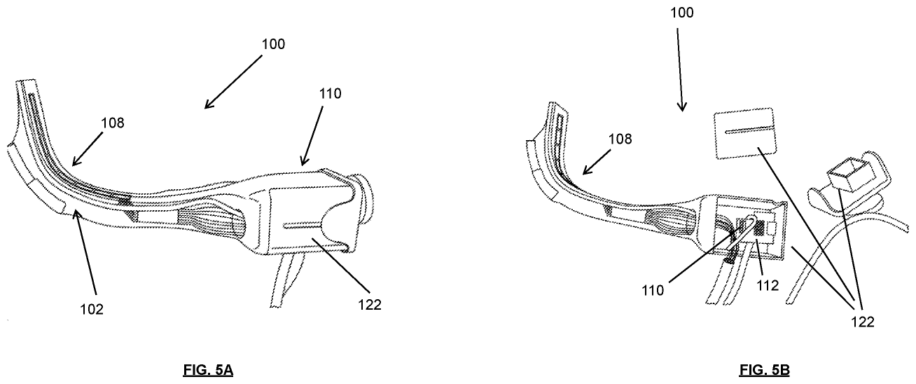

Referring to FIGS. 3, 4, 5A and 5B, different embodiments of an instrumented or force-sensor equipped laryngoscope 100 are illustrated. In FIG. 3, the laryngoscope comprises a handle 104, a blade 102 and a hinge or connecting base 106. The tongue-contacting portion, or inward face of the blade 102, is provided with a series of individual force sensors 108i to 108viii, spaced-part along the length of the blade, such as load cells. In FIG. 4, the force sensors 108i-viii are provided on a flexible sensor strip, positioned on the inner surface of the blade 102. Each force sensor connects back to an onboard PCB 110, which can be located inside a casing in or on the blade, in the hinge connection 106 or in the handle 114. The PCB unit comprises one or more input ports or connections for receiving the respective force signals from the force sensors, and one or more output ports or connections for sending force data via a wired or wireless link to the processing device 200. While hidden on FIG. 4, the PCB includes a communication unit or module which manages the flow of force data being sent for further processing. As mentioned above, force signal processing can be conducted by a microprocessor on the PCB and/or sent to a remote processing device via the communication link 114. Thus, in possible embodiments, it can be considered to have the processing capacity provided solely on the laryngoscope, or distributed between chips on the laryngoscope, and the processing device 200, which can be a computer, laptop, tablet, phone or server. The processing device can be a single device 200 locally or remotely located from the laryngoscope or distributed between several sub-units or processing devices. For example, the reference force distribution data can be stored in a storage memory provided on a chip on the laryngoscope or be stored remotely, for example on a computer or server 20, in a lookup table. The comparison between the force distribution being applied and the reference force distribution (determined while an expert performed a laryngoscopy for example) can be performed based on predetermined thresholds or using machine learning and trained predictive models, as will be explained in greater detail below. FIGS. 5A and 5B illustrate yet another embodiment of an instrumented laryngoscope with force sensors provided along the blade, with the PCB protected in a PCB housing or protective casing 122.

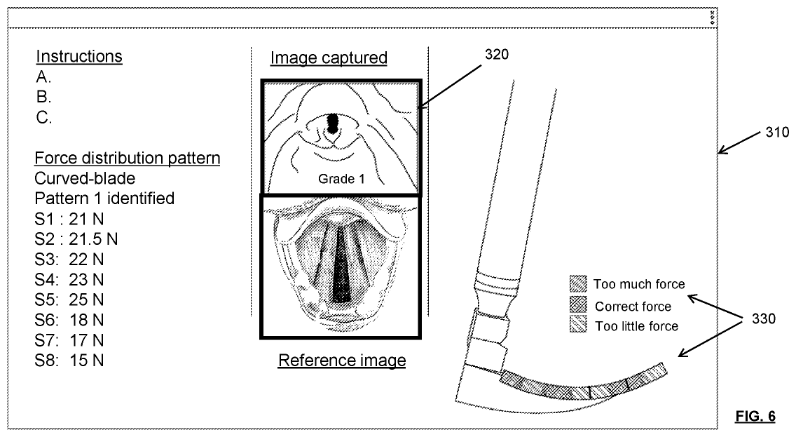

Referring now to FIG. 6, an exemplary graphical user interface 310 is illustrated. The graphical user interface 310 comprises, on the right-side pane, a visual representation 330 of the blade, wherein the force distribution and/or the deviation is illustrated on the blade, preferably in real-time, as the user manipulates the blade. The deviation need not be illustrated on the blade, and in other embodiments, the indication 330 of the force distribution and/or of the force distribution deviation could be located near the blade, or provided in a table or by any other representation, using color, shapes, icons, letters or numbers. The graphical user interface 310 thus provides feedback to the user regarding force adjustments to be made along the blade when performing the laryngoscopy, for example by indicating on which portions of the blade too much, too little or enough/adequate force is being applied. The capacity of determining the force distribution advantageously allows assessing where and how much force is being applied, to limit potential damage to internal airway structures, by applying an improper force distribution. The force distribution also allows differentiating different force patterns used by trainees, such as cranking toward teeth, wrong lifting, etc., and recognize if the intubation is being conducted properly or not and how far trainees are from an expert pattern.

Still referring to FIG. 6, and also to FIG. 2, in possible embodiments, the processing device 200 can comprise or have access to a database for storing the current and previously recorded force data. The processing device 200 may also comprise, or have access to, a trained predictive model 302 to recognize or detect the force distribution pattern being applied by the user based on previously learned patterns derived from the previously recorded force data. The trained predictive model can be stored in the memory of the processing device, as part of the laryngoscopy software training application 300, to provide, via the graphical user interface 310, personalized feedback 330 to the user with regard to force adjustments needed to come closer to one of the previously learned patterns. The trained predictive model can also provide an indication of the likelihood or probability that the force distribution applied by the user on the blade of the laryngoscope corresponds (or is similar) to a given force pattern. A force pattern can be associated with a given insertion technique or with a distribution of the forces along the blade.

Still referring to FIG. 6, a video image 320 is also shown. As schematically represented on FIG. 2A, a video camera 116 can be provided on the blade 102, and the images or video stream 320 captured during the laryngoscopy can be displayed on the graphical user interface 310, together with the force distribution 330 being applied on the blade. A target or reference image of a larynx can also be shown, corresponding to an image captured while an expert performed a successful laryngoscopy, in addition to the current/real-time image resulting from the user manipulating the laryngoscope. Video laryngoscopes have been used clinically but remain rarely used for training purposes because of their unavailability, since there is usually only a small number of units in hospitals due to their high investment cost. This leads to laryngoscopy and intubation being taught with direct laryngoscopy given the inability of trainers to see what trainees are looking at inside the airway since there is no camera. Adding an on-board video camera 116 to the training laryngoscope 100 allows providing more personalized feedback to users. In addition to providing users with feedback on the force distribution they are applying, the platforms 500 shows, via the graphical user interface, a live video capture while a user manipulates the training laryngoscope 100. Just as for determining the force distribution being applied, and how far/close it is from a reference force distribution pattern, a predictive model/machine learning algorithm can be used to assess if a proper glottic view is achieved in order to provide accurate diagnostics for future intervention according to different training scenarios.

In other possible embodiments, the tip 120 can comprise the on-board video camera 116. In such case, the laryngoscopy training application 300 can further comprise an additional trained predictive model 302i, trained on previously captured laryngoscopy images labelled as valid or invalid, to determine whether the tip of the blade is properly positioned, based on reference images previously captured and classified. In yet other embodiments, this additional trained predictive model 302i can further determine a grade or degree of aperture (in % or in units of area) of the larynx based on previously labelled laryngoscopy images.

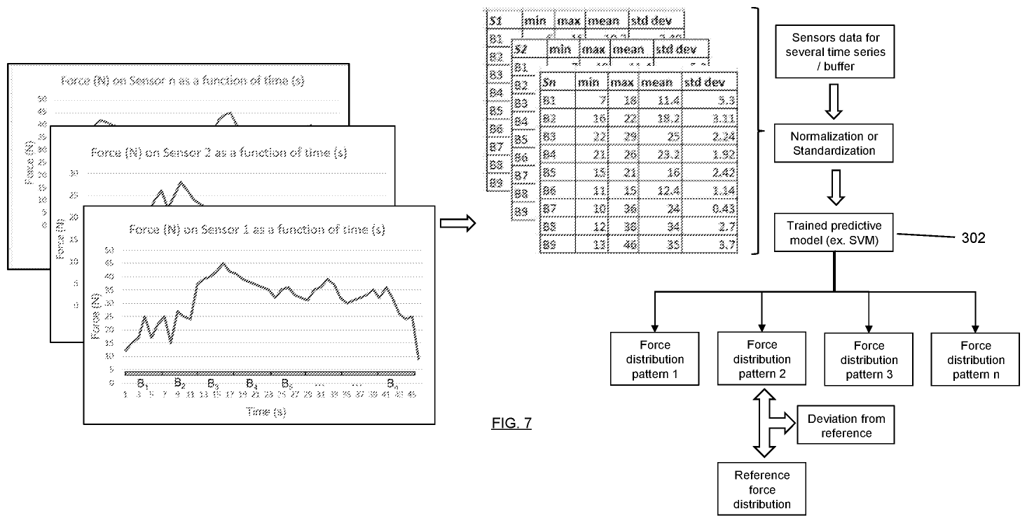

Referring now to FIG. 7, in addition to FIGS. 2, 2A and 6, in a possible embodiment, the laryngoscopy training application 300 comprises a trained predictive model 302. In the example, the trained predictive model 302 is a Support Vector Machine (SVM) model, but other predictive models can be used as well, such as neural network or decision tree models. The trained predictive model can be a supervised learning model configured to analyze data for classification or to conduct a regression analysis. For each sensor of the laryngoscope 108-108n, the force applied thereto is measured and recorded as a function of time, throughout the laryngoscopy training procedure, as schematically represented by the graphs on the left side of FIG. 7. Time series or buffers (B1 to Bn) are recorded, wherein a time series comprises force readings made by the sensor during a predetermined buffer period, such as 5 seconds in the example. The time buffers (B1 to Bn) may overlap or not, and preferably have the same period. For each sensor, and for each time buffer, statistical data is calculated, such as the minimum force value, the maximum force value, the average force value and the standard deviation force value. This statistical data can be processed, such as normalized or standardized. For example, all statistical parameters can be normalized by being converted to be within a range of 0 to 1, or standardized with the mean centered around 0 and the standard deviation set to 1, as possible normalization or standardization processes. The normalized or standardized force data is then fed to the trained predictive model (302), to determine which type of force distribution pattern the user manipulating the laryngoscope is applying. For example, the force distribution pattern may vary depending on the type of laryngoscope (curved vs straight blade) and depending on the insertion technique being used. This force distribution pattern can be compared to a reference force distribution pattern (based on an expert performing an “ideal” or “standard” laryngoscopy), and feedback indicating whether too little, correct or adequate, or too much force is being applied to each of the portions of the blade, is provided to the user, via the graphical user interface. For example, an indication that the force being applied is insufficient or excessive on a given portion or segment of the blade can be displayed if the force detected on that given blade portion is below or above force thresholds, the thresholds being set based on ideal or reference force distributions.

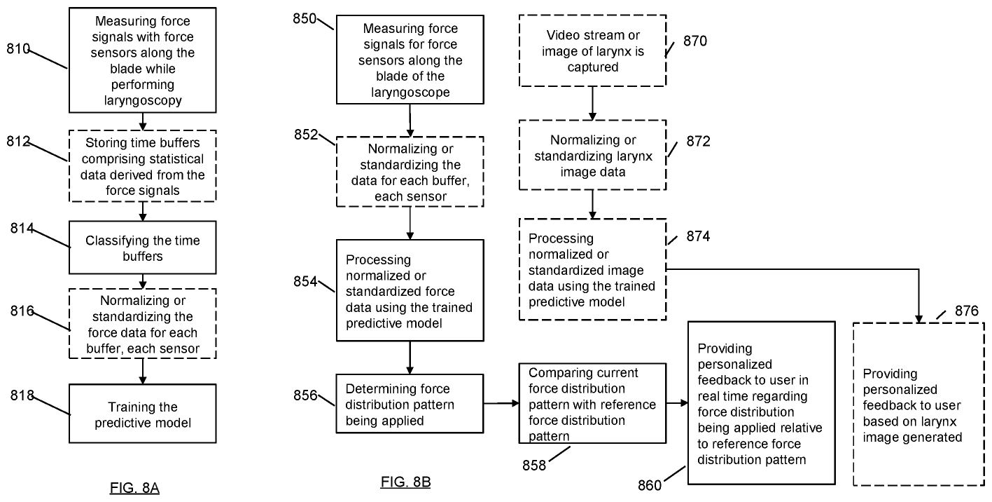

Referring to FIG. 8A, the general steps for training the predictive model, according to a possible embodiment, are schematically illustrated in a flow chart. Forces are continuously measured by each of the sensors disposed along the blade (steps 810) during a predetermined period while an instructor or clinician performs a laryngoscopy using the instrumented laryngoscope. The force measurements are stored on the storage medium, including for example a memory on a PCB, in a laptop or remote server, and divided in time buffers (step 812). The time buffers can be labelled or classified individually, or alternatively, the force distribution for the entire time interval can be labelled and classified. Classification can relate to the type of laryngoscope being used, the laryngoscope insertion technique being applied and/or to force distribution patterns. A measured force distribution can be classified as valid or invalid based on its similarity with reference force distribution patterns. For each buffer, statistical data is calculated, and then normalized or standardized (as explained with reference to FIG. 7). The predictive model (in the example: an SVM model) can then be trained with the labelled force data to be able, for example, to distinguish force distribution patterns when users will train or use the laryngoscopy platform 500.

Referring now to FIG. 8B, when used during a training and/or an evaluation of the performance of a user while manipulating the instrumented laryngoscope, force signals are continuously or periodically measured (step 850), for each sensor. Time series or buffers are used, so at to provide real time feedback to the user. Statistical force data is derived from the force measurements, and this force data is normalized and standardized (step 852). The force data is then processed through the trained predictive model (step 854), which can determine the force distribution pattern being applied (step 856). The force distribution pattern can then be compared to one or more reference force distribution patterns previously determined as valid (step 858) and provide personalized feedback to the user. The graphical user interface can also further indicate to the user which force distribution pattern is being applied by the user. By “comparing”, it is meant that the trained predictive model receives force data or a force distribution and is trained to predict or assess a similarity thereof with a reference force distribution. The assessment can be provided per zone or areas on the blade of the laryngoscope. The zones or areas can be distributed along the blade, such as from the tip to the base of the blade.

Optionally, several force distribution patterns associated to a user can be stored over time. The trained predictive model can in this case provide an indication, for output to the graphical user interface, of the improvement of the user's performance over time, in reaching a standard force distribution pattern, comprising a step of determining additional practice time required by the user to reach a standard force distribution pattern using the predictive model. The improvement in user performance can be indicated by showing the increase in similarity between the measured force distribution and the reference force distribution. The laryngoscopy training application, using the predictive model, can thus be configured to determine whether—and how much—additional practice time is required by the user to reach a standard force distribution pattern. Existing training systems do not provide relevant data as to how experts are doing compared to other experts and how often they should undergo recurring training for continuous improvement.

Still referring to FIG. 8B, the training platform can be further adapted and configured such that a video stream or an image can be captured (step 870) while the user or trainee positions the blade in the airway of the patient or manikin. The image or stream of images data is normalized or standardized (step 872) and can be fed to a trained predictive or machine learning model (step 874), such as a convolutional neural network (CNN) to compare the image being captured with previously captured images determined as valid, to further guide the user as to whether the blade is properly positioned in the larynx (step 876).

According to a possible implementation of the training platform, a server database or storage medium compiling all experts training results allows the predictive model and related algorithms to analyze different force measurement (and possibly blade position) datasets and provide information regarding how well users are progressing toward becoming an expert and how often they should practice to keep skills sharp.

Several alternative embodiments and examples have been described and illustrated herein. The embodiments of the invention described above are intended to be exemplary only. A person skilled in the art would appreciate the features of the individual embodiments, and the possible combinations and variations of the components. A person skilled in the art would further appreciate that any of the embodiments could be provided in any combination with the other embodiments disclosed herein. It is understood that the invention may be embodied in other specific forms without departing from the central characteristics thereof. The present examples and embodiments, therefore, are to be considered in all respects as illustrative and not restrictive, and the invention is not to be limited to the details given herein. Accordingly, while specific embodiments have been illustrated and described, numerous modifications come to mind without significantly departing from the scope of the invention.

1. A laryngoscopy training system, comprising:
a laryngoscope including a blade provided with a plurality of force sensors therealong, the force sensors being adapted to detect forces applied thereon resulting from a user inserting or manipulating the laryngoscope inside a mouth and an airway, and to transmit respective force signals,
a processing device comprising a processor and storage medium having stored thereon processor-readable instructions for processing force data derived from the respective force signals, determining a force distribution along the blade based on the force data; comparing the force distribution determined with a reference force distribution; and outputting for display a visual indication indicative of a deviation of the force distribution being applied along the blade from the reference force distribution, in real-time.

2. The laryngoscopy training system of claim 1, wherein the force sensors are provided on a flexible sensor strip positioned on an inner surface of the blade devised to be in contact with airway structures.

3. The laryngoscope of claim 1, further comprising a display for displaying a graphical user interface, the graphical user interface comprising the visual indication.

4. The laryngoscopy training system of claim 1, further comprising a printed circuit board (PCB) including a communication unit, the communication unit including input connections for receiving the respective force signals from the force sensors, and one or more output connection(s) for sending the force data via a wired or wireless connection to the processing device.

5. The laryngoscopy training system of claim 1, wherein reference force distribution data is stored onto the storage medium of the processing device and the comparing is performed based on predetermined thresholds.

6. The laryngoscopy training system of claim 1, wherein the visual indication comprises a representation of the blade, and wherein the force distribution and/or the deviation is illustrated on or near the blade using color, icons, letters or numbers.

7. The laryngoscopy training system of claim 3, further comprising an on-board video camera provided on or near the blade, for capturing images during training sessions, the graphical user interface further displaying the images captured in real-time, in addition to the visual indication of the force distribution and/or deviation from the reference force distribution.

8. The laryngoscopy training system of claim 1, wherein:
the processing device comprises a trained predictive model for recognizing a force distribution pattern applied by the user based on previously learned force distribution patterns derived from previously recorded force data,
the processing device being further configured for providing for output personalized feedback to the user regarding force adjustments needed to come closer to one of the previously learned force distribution patterns.

9. The laryngoscopy training system of claim 8, wherein:
the processing device is configured for storing several force distribution patterns associated with a user over time; and
the processing device is further configured for providing, using the trained predictive model, an indication of an improvement of a performance of the user over time, in reaching a standard force distribution pattern.

10. The laryngoscopy training system of claim 7, wherein
the processing device further comprises an additional trained predictive model trained on previously captured laryngoscopy images labelled as valid or invalid, to determine whether the blade is properly positioned.

11. The laryngoscopy training system of claim 10, wherein the additional trained predictive model can further determine a grade or degree of aperture of the larynx based on previously labelled laryngoscopy images.

12. The laryngoscopy training system of claim 8, wherein the processing device is configured for processing in real time a plurality of time buffers, and for computing, for each time buffer, statistical data of the forces measured by each of the sensors during a predetermined period while an instructor or clinician performs a laryngoscopy using the laryngoscope, the trained predictive model being configured to detect force distribution patterns along the blade using the statistical data computed for the plurality of time buffers.

13. The laryngoscopy training system of claim 8, wherein the trained predictive model is a support-vector machine (SVM) model.

14. The laryngoscopy training system of claim 8, comprising a step of associating the distribution of forces applied by the user to a given force distribution pattern determined using a predictive model.

15. The laryngoscopy training system of claim 8, comprising determining additional practice time required by the user to reach a standard force distribution pattern using the predictive model.

16. A method for evaluating a performance of a user in performing a laryngoscopy, the method comprising:
measuring force signals associated to forces applied to different portions of a blade of a laryngoscope manipulated by a user during the laryngoscopy;
converting the force signals into force data indicative of a distribution of forces along the blade;
comparing the distribution of forces along the blade with at least one previously determined force distribution pattern; and
providing for output, in real time, an indication of whether too little, adequate or too much force is applied to each of the different portions of the blade, relative to the at least one previously determined force distribution pattern, while the user manipulates the laryngoscope.

17. The method of claim 16, wherein comparing the distribution of forces is performed using a predictive model trained on previously collected force signals, collected during a valid laryngoscopy procedure.

18. The method of claim 16, comprising determining additional practice time required by the user to reach a standard force distribution pattern using the predictive model.

19. A non-transitory storage medium for storing processor-executable instructions for evaluating a performance of a user in performing a laryngoscopy, the instructions causing a processor to:
process force data derived from force signals indicative of forces applied to different portions of a blade of a laryngoscope manipulated by a user during the laryngoscopy determine a distribution of forces along the blade;
compare the distribution of forces along the blade with at least one previously determined force distribution pattern; and
provide for output, in real time, an indication of whether too little or too much force is applied to each of the portions of the blade, relative to the at least one previously determined force distribution pattern, while the user manipulates the laryngoscope.

20. The non-transitory storage medium of claim 19, further comprising instructions for causing the processor to:
use a predictive model trained on previously collected force signals, collected during a valid laryngoscopy procedure.

21. The non-transitory storage medium of claim 19, further comprising instructions for causing the processor to:
determine additional practice time required by the user to reach a standard force distribution pattern using the predictive model.
